(19) United States
(12) Patent Application Publication
(10) Pub. No.: US 2019/0167930 A1
(43) Pub. Date: Jun. 6, 2019

(54) Methods and Systems for the Treatment of Sleep Apnea

(71) Applicant: Fresca Medical Inc., San Clemente, CA (US)

(72) Inventors: John Cox, San Clemente, CA (US); Ed McCarthy, San Clemente, CA (US); Richard Ewers, Fullerton, CA (US)

(21) Appl. No.: 16/210,697

(22) Filed: Dec. 5, 2018

Related U.S. Application Data
(60) Provisional application No. 62/595,529, filed on Dec. 6, 2017.

(51) Int. Cl. A61M 16/00; A61B 5/00

(52) U.S. Cl. CPC A61M 16/0066; A61B 5/4818; A61F 5/56; A61M 2016/0036; A61M 16/024

(57) ABSTRACT

A blower for the treatment of a patient's sleep apnea is disclosed. The blower includes a start/stop button, an air pump, and a processor connected to the start/stop button and air pump. A user may input a snooze period (Tsnooze), a ramp period, (Tramp1), and an ultimate therapeutic pressure (TP). The processor performs an snooze function that allows a user to stop the delivery of pressure from the blower for the snooze period, after which the processor ramps the pressure gradually over the ramp period until plateauing at the ultimate therapeutic pressure. The processor may limit the number of snooze triggers that a patient may use.

### RELATED APPLICATIONS

The assignee of this application, FRESCA Medical, has described various embodiments of its valved Positive Airway Pressure (PAP) sleep apnea treatment mask. Those embodiments are described in U.S. patent application Ser. No. 13/860,926, filed Apr. 11, 2013, titled “Sleep Apnea Device,” U.S. Provisional Application Ser. No. 61/623,855, filed Apr. 13, 2012, titled “Sleep Apnea Device,” U.S. Provisional Application Ser. No. 61/775,430, filed Mar. 8, 2013, titled “Sleep Apnea Device,” U.S. Provisional Application No. 61/823,553, filed May 15, 2013, titled “Sleep Apnea Device,” U.S. Provisional Application No. 61/838,191, filed Jun. 21, 2013, titled “Sleep Apnea Device,” U.S. Provisional Application No. 61/962,501, filed Nov. 8, 2013, titled “Sleep Apnea Device,” U.S. Provisional Application No. 61/909,956, filed Nov. 27, 2013, titled “Sleep Apnea Device,” U.S. Provisional Application No. 61/927,355, filed Jan. 14, 2014, titled “Valve with Pressure Feedback,” U.S. Provisional Application No. 62/134,506 filed Mar. 17, 2015 titled “Valve with Pressure Feedback Draft Provisional Application,” U.S. Provisional Application No. 62/163,601, filed May 19, 2015, titled “Airflow Generator with Delayed Onset”, U.S. Provisional Application No. 62/184,787 filed Jun. 25, 2015 titled “Sleep Apnea Device,” U.S. Provisional Application No. 62/239,146 filed Oct. 8, 2015 titled “Sleep Apnea Device,” U.S. patent application Ser. No. 14/930,284, filed Nov. 2, 2015, titled “Apparatus, System and Methods for Treating Obstructive Sleep Apnea”, U.S. Provisional Application No. 62/246,339 filed Oct. 26, 2015 titled “Venting of a Valved CPAP Mask to Create a Comfortable Breathing Sensation”, U.S. Provisional Application No. 62/246,489 filed Oct. 26, 2015 titled “Managing Sleep Apnea with Pulse Oximeters and With Additional Assessment Tools”, U.S. Provisional Application No. 62/246,328 filed Oct. 26, 2015 titled “Novel Low Flow Technology Designed to Meet CPAP Efficacy”, U.S. Provisional Application No. 62/246,477 filed Oct. 26, 2015 titled “Composite Construction Air Delivery Hose for Use with CPAP Treatment”, U.S. Provisional Application No. 62/275,899 filed Jan. 7, 2016 titled “Valved Mask To Reduce and Prevent Snoring”, U.S. Provisional Application No. 62/311,804 filed Mar. 22, 2016 titled “Improvements to Sleep Apnea Machine”, U.S. Provisional Application No. 62/382,980 filed Sep. 2, 2016 titled “Dual Rotatable Hose For Use With CPAP Treatment”, U.S. application Ser. No. 15/334,243 filed Oct. 15, 2016 titled “Apparatus, Systems, and Methods For Treating Obstructive Sleep Apnea”, U.S. Provisional Application No. 62/532,240 filed Jul. 13, 2017 titled “Sleep Apnea Treatment System and Improvements Thereto”, U.S. patent application Ser. No. 15/557,907 filed on Sep. 13, 2017 titled “Apparatus, Systems, and Methods For Treating Obstructive Sleep Apnea”, U.S. Provisional Application No. 62/465,905 filed Mar. 2, 2017 titled “Sound Mitigation/Flow Optimization in a Valved Obstructive Sleep Apnea Treatment Mask”, U.S. patent application Ser. No. 16/034,980 filed on Jul. 13, 2018 titled “Sleep Apnea Treatment System and Improvements Thereto”, U.S. patent application Ser. No. 16/034,967 filed on Jul. 13, 2018 titled “Sleep Apnea Treatment System and Improvements Thereto”, U.S. Provisional Application No. 62/722,580 filed on Aug. 24, 2018 titled “Braided Hose For Use in Sleep Apnea Treatment Systems that Decouples Forces”, U.S. Provisional Application No. 62/686,442 filed on Jun. 18, 2018 titled “Braided Hose For Use in Sleep Apnea Treatment Systems that Decouples Forces”, U.S. Provisional Application No. 62/694,126 filed on Jul. 5, 2018 titled “Braided Hose For Use in Sleep Apnea Treatment Systems that Decouples Forces”, U.S. patent application Ser. No. 15/557,907 filed on Sep. 13, 2017 titled “Apparatus, Systems, and Methods For Treating Obstructive Sleep Apnea”, and PCT/US16/23798 titled “Apparatus, Systems, and Methods For Treating Obstructive Sleep Apnea” filed on Mar. 23, 2016, all of which are hereby incorporated by reference in their entirety. Disclosed in this document are particular features and structures that may be used in conjunction with the previously disclosed embodiments.

This application further claims priority as the non-provisional of U.S. Provisional Application No. 62/595,529 filed Dec. 6, 2017 titled “Sleep Apnea Treatment System and Improvements Thereto”, the entire contents of which is hereby incorporated by reference.

### TECHNICAL FIELD

The present invention is related to medical systems, devices, and methods. More specifically, the invention is related to systems, devices and methods for treating obstructive sleep apnea or snoring.

### BACKGROUND

Obstructive sleep apnea (OSA) is a common medical disorder that can be quite serious. It has been reported that approximately one in twenty-two Americans (about 12,000,000 people) suffer from OSA, and many cases go undiagnosed. Chronic fatigue has long been recognized as the hallmark of OSA, but more recently, large clinical studies have shown a strong link between OSA, strokes and death.

Obstructive sleep apnea is a condition in which the flow of air pauses or decreases during breathing while one is asleep, because the airway has become narrowed, blocked, or floppy. A pause in breathing is called an apnea episode, while a decrease in airflow during breathing is called a hypopnea episode. Almost everyone has brief apnea or hypopnea episodes while they sleep. In OSA, however, apnea episodes occur more frequently and last longer than in the general population. OSA has become an increasingly costly medical condition in recent years, as the disorder is more prevalent in obese people and obesity has become significantly more prevalent. Unfortunately, the currently available options for treating OSA are not ideal.

A person with OSA usually begins snoring heavily soon after falling asleep. Often the snoring gets louder. The snoring is then interrupted by a long silent period during which there is no breathing. This is followed by a loud snort and gasp, as the person attempts to breathe. This pattern repeats. Many people wake up unrefreshed in the morning and feel sleepy or drowsy throughout the day. This is called excessive daytime sleepiness (EDS). People with sleep apnea may act grumpy or irritable, be forgetful, fall asleep while working, reading, or watching TV, feel sleepy or even fall asleep while driving, or have hard-to-treat headaches. OSA sufferers may also experience depression that becomes worse, hyperactive behavior (especially in children), or leg swelling (if severe).

The most widely used therapy for OSA is Positive Airway Pressure (PAP). A PAP system typically consists of a mask fitting in or over the nose or nose and mouth, an air pressurizing console (or blower) and a hose connecting the two (typically a six-foot long hose with a 20 mm diameter bore). PAP works by pressurizing the upper airway throughout the breathing cycle, essentially inflating the airway to keep it open and thus creating what is sometimes referred to as a “pneumatic splint.” This flow is at set pressure that has been predetermined through medical testing to be appropriate to create a pneumatic splint in the user's airway. This prevents airway collapse and allows the user to breath without obstruction. Because the masks typically leak air, PAP systems have to provide an airflow rate of up to 200 liters per minute (approximate figure based on unpublished data). The high airflow rate is needed for multiple reasons. First, all the air needed for breathing must come through the hose. Second, conventional masks have an intended leak built in for the purpose of constant “CO2 washout.” Third, these systems achieve the required pressure by using a high airflow rate to generate a back-pressure at the mask end where the air is leaking out. Unfortunately, this high flow rate makes breathing feel quite uncomfortable for many users and requires a relatively large, noisy blower. Additionally, the high required flow rates of PAP often cause discomfort during exhalation due to increased resistance, as well as nasal dryness, dry mouth, ear pain, rhinitis, abdominal bloating and headaches.

The overwhelming shortcoming of PAP is poor user compliance. Over half of all users who try PAP stop using it. Patients tend to abandon therapy at an alarming rate, at rates of between 25-50% in the first year. Users dislike the side effects mentioned above, as well as having to wear an uncomfortable, claustrophobia inducing mask, being tethered to a pressurizing console, the noise of the console, traveling with a bulky device, and a loss of personal space in bed. Many attempts have been made to add features to PAP therapy to help patients with acclimation.

Many PAP devices and alternatives to PAP have been developed, but all have significant shortcomings. Less invasive attempts at OSA treatment, such as behavior modification, sleep positioning and removable splints to be worn in the mouth, rarely work. A number of different surgical approaches for treating OSA have also been tried, some of which are still in use. For example, Uvulopalatopharyngoplasty (UPPP) and Laser Assisted Uvula Palatoplasty (LAUP) are currently used. Surgical approaches, however, are often quite invasive and not always effective at treating OSA.

One alternative approach to OSA treatment is to provide a pneumatic splint during the expiratory portion of the respiratory cycle by producing a partial blockage in the nose or mouth, thus slowing the release of air during expiration and increasing positive pressure in the airway. The simplest way to form an expiratory pneumatic splint, pursing the lips, has been shown to open the upper airway and improve breathing in emphysema users. This type of maneuver is generically labeled Expiratory Positive Airway Pressure (EPAP).

Therefore, it would be advantageous to have improved systems, devices and methods for treating OSA and snoring. Ideally, such systems, devices and methods would be less cumbersome than currently available PAP systems, help to improve user compliance. Also ideally, such systems, devices and methods would provide some of the advantages of an expiratory pneumatic splint. At least some of these objectives were met by the embodiments described in references listed above and incorporated herein by reference.

While these references are an important improvement over the state of the art, it would be advantageous to improve upon these systems by increasing user compliance and comfort.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

Provided in various example embodiments is an improved blower for the treatment of a patient's sleep apnea is disclosed. The blower includes a start/stop button, an air pump, and a processor connected to the start/stop button and air pump. A user (e.g. a patient or a physician) may input a sleep period (Tsleep), an ultimate therapeutic pressure (TP) and an acclimation period (Nset). Tsleep may be at a default of 4 hrs/night, which is the clinically accepted standard for a night's compliant use. The processor performs an acclimation procedure to ease the patient in to the use of the positive airway pressure (PAP) system over the selected acclimation period. Specifically, the processor (a) sets Tsleep, T P, and Nset, along with setting a day counter (Nday). Next, the processor (b) determines when the patient presses the start/stop button and then it (i) begins a timer (T), (ii) sets the air pump to deliver air pressure at a pressure based on ((Nday/Nset)×TP), and (iii) when the patient presses start/stop button again, the processor pauses T, stops air pressure delivery, and determines if the patient has sleep long enough (i.e., comparing T to Tsleep), and if so if the number of acclimation days has been exceeded (i.e., Nset<Nday). If the patient has not slept long enough (i.e., T<Tsleep) then the processor returns to waiting until the patient presses the start/stop button again (step b). If the patient has slept long enough but the acclimation days have not been exceeded, then the processor increases Nday, resets T and returns to waiting until the patient presses the start/stop button again (step b). If the patient has slept long enough and has exceeded the acclimation days, then the processor exits the acclimation procedure.

The acclimated daily air pressure (i.e., (Nday/Nset)×TP)), may include a ramp period, over which the pressure is gradually incremented to increase patient comfort, and a pre-ramp period during which no pressure is provided. In such an embodiment, a processor controls ramp period (Tramp1) and a pre-ramp period (Tpr). Instead of providing an immediate acclimated daily air pressure (i.e., (Nday/Nset)×TP)) upon the patient pressing the start stop button, the processor first allows Tpr to elapse and then sets the air pump to deliver air pressure at a pressure based on ((Nday/Nset)×(T2/Tramp1)×TP). Once the Tramp has elapse, the air pressure deliver is plateaued at the acclimated daily air pressure (i.e., (Nday/Nset)×TP)).

The processor may set Tsleep, TP, Nset, Tramp1, and Tpr based on the user's (e.g. patient or physician) input. Optimal values for Tsleep is between 4 and 8 hours, for TP is between 4 and 20 CM H20, for Nset is between 3 and 14 days, and for either or both of Tramp1 and Tpr is between 0.25 and 1.5 hours.

In a second embodiment, a user (e.g. a patient or a physician) may input a snooze period (Tsnoze), a ramp period, (Tramp1), and an ultimate therapeutic pressure (TP). The processor performs a snooze function to allow a patient in discomfort to temporarily postpone therapeutic pressure from the blower box. Specifically, the processor (a) controls Tsnooze, Tramp1, and TP. Next, the processor (b) determines when the patient presses the start/stop button and then it (i) allows Tsnooze to elapse, starts a timer (T3), and set the air pump to deliver air pressure at a pressure based on ((T3/Tramp1)×TP). If during step (b)(i) the processor detects that the patient has pressed the start/stop button, then the processor resets T3, stops delivering air pressure, and return to step (b)(i).

The air pressure delivered to the patient may be plateaued at TP when the ramp period has elapsed (i.e., T3 is >=Tramp1). If the during the plateau the processor detects that the patient has pressed the start/stop button, then the processor resets T3, stops delivering air pressure, and return to step (b)(i).

The processor may also check if the user has used the system long enough such that the pressing of the start/stop button is no longer considered a snooze trigger. To implement this, the processor sets a snooze disable period (Tds) and starts a timer (T1). If the processor detects that the patient has pressed the start/stop button, but only after the snooze disabled period has elapsed (i.e., Tds>=T1) then the processor stops the blower box.

The processor may also implement a pre-snooze period (Tps), such that when the patient presses the snooze, the system does not take as long to begin the ramp up in pressure, as it does when the blower is initially turned on. To implement this, the processor sets a pre-snooze period (Tps) and after step (b) but before (b)(i), the processor allows Tps to elapse. Any subsequent snooze triggers by the patient would, therefore cause the processor to re-start the method after the Tps has elapsed.

If a patient over-utilizes the snooze trigger, then the patient will not gain the benefit of the therapeutic pressure from the blower box. To prevent this, the processor sets a maximum number for snooze triggers (Nmax) and initially sets a counter N=0. Each time the processor detects that the patient has pressed the start/stop button, the processor increases N. When N exceeds the maximum number for snooze triggers (i.e., N>=Nmax), then the processor will ignore any subsequent snooze triggers.

If a patient has already reached the ultimate therapeutic pressure, then it is likely that the patient is comfortable enough to reach that pressure with a shorter ramp period should the patient trigger a snooze. Therefore, the processor may set a second ramp period (Tramp2) that is shorter than the ramp period (Tramp1), when the patient has already reached that the ultimate therapeutic pressure. And during Tramp2, the processor sets the air pump to deliver air pressure at a pressure based on ((T3/Tramp2)×TP) until Tramp2 elapses, at which point the pressure is plateaued at TP.

The processor may set Tsnooze, Tramp1, Tramp2, Tds, Tps, TP and Nmax based on the user's (e.g. patient or physician) input. Optimal values for Tsnooze, Tramp 1, Tramp 2 and Tps is between 0.1 and 1.5 hours, for Tds is between 4 and 8 hours, TP is between 4 and 20 CM H20, for Nmax is between 3 and 6.

In a third embodiment, the processor may implement both the snooze and acclimation functions discussed above.

Additional aspects, alternatives and variations as would be apparent to persons of skill in the art are also disclosed herein and are specifically contemplated as included as part of the invention. The invention is set forth only in the claims as allowed by the patent office in this or related applications, and the following summary descriptions of certain examples are not in any way to limit, define or otherwise establish the scope of legal protection.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure. It will be understood that certain components and details may not appear in the figures to assist in more clearly describing the invention.

FIG. 1A illustrates a PAP system.

FIG. 1B illustrates some components of a PAP blower box.

FIG. 2A illustrates a blower used as part of a PAP, wherein the blower is in a snooze configuration.

FIG. 2B illustrates a blower used as part of a PAP, wherein the blower is no longer in a snooze configuration.

FIG. 2C illustrates a blower used as part of a PAP, wherein the blower has disabled snooze.

FIG. 3A illustrates a pressure over time curve for PAP system implementing an acclimation period on the first day.

FIG. 3B illustrates a pressure over time curve for PAP system implementing an acclimation period on day subsequent to the one shown in FIG. 3A.

FIG. 3C illustrates a pressure over time curve for PAP system implementing an acclimation period on the last day.

FIG. 4A is a flow chart showing the steps for an acclimation method to be implements in a PAP system.

FIG. 4B is a flow chart for a subroutine that may be used in conjunction with the method illustrated in FIG. 4A.

FIG. 5A illustrates a pressure over time curve for a system implementing a snooze function.

FIG. 5B illustrates a pressure over time curve for a system implementing a snooze function.

FIG. 6A is a flow chart showing the steps for a snooze method to be implements in a PAP system.

FIG. 6B is a flow chart for a subroutine that may be used in conjunction with the method illustrated in FIG. 6A.

FIG. 6C is a flow chart for a subroutine that may be used in conjunction with the method illustrated in FIG. 6A.

FIG. 7A is a flow chart showing the steps for a snooze and acclimation method to be implements in a PAP system.

FIG. 7B is a flow chart for a subroutine that may be used in conjunction with the method illustrated in FIG. 7A.

### DETAILED DESCRIPTION OF EXAMPLE EMBODIMENTS

Reference is made herein to some specific examples of the present invention, including any best modes contemplated by the inventor for carrying out the invention. Examples of these specific embodiments are illustrated in the accompanying figures. While the invention is described in conjunction with these specific embodiments, it will be understood that it is not intended to limit the invention to the described or illustrated embodiments. To the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, process operations well known to persons of skill in the art have not been described in detail in order not to obscure unnecessarily the present invention. Various techniques and mechanisms of the present invention will sometimes be described in singular form for clarity. However, it should be noted that some embodiments include multiple iterations of a technique or multiple mechanisms unless noted otherwise. Similarly, various steps of the methods shown and described herein are not necessarily performed in the order indicated, or performed at all in certain embodiments. Accordingly, some implementations of the methods discussed herein may include more or fewer steps than those shown or described. Further, the techniques and mechanisms of the present invention will sometimes describe a connection, relationship or communication between two or more entities. It should be noted that a connection or relationship between entities does not necessarily mean a direct, unimpeded connection, as a variety of other entities or processes may reside or occur between any two entities. Consequently, an indicated connection does not necessarily mean a direct, unimpeded connection unless otherwise noted.

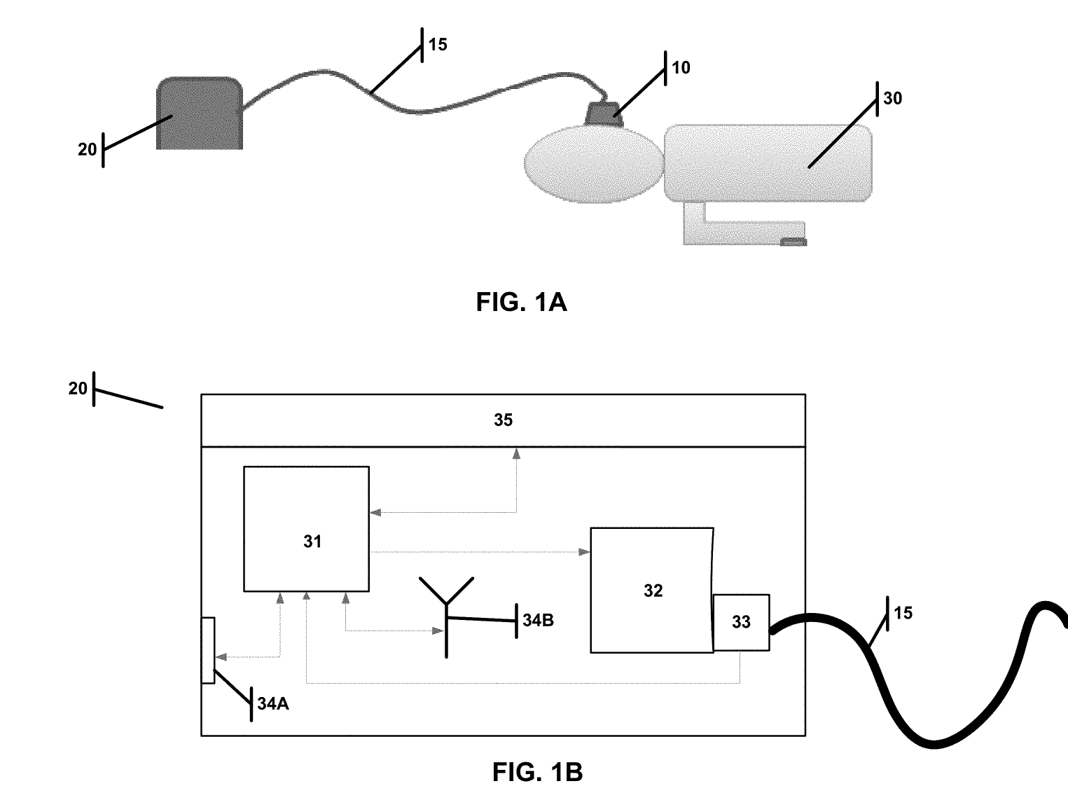

The following list of example features corresponds with FIGS. 1-7B and is provided for ease of reference, where like reference numerals designate corresponding features throughout the specification and figures:
Sleep apnea mask 10
Hose 15
Blower box 20
Patient 30
Processor 31
Air pump 32
Flow sensor 33
Card reader 34A
Antenna/Transceiver 34B
Blower display/touchscreen 35
Snooze start/stop button 40
Current blower setting 45
Stop button 50
Stop button after snooze disabled 55
Snooze disabled notification 57
Pressure/time curve for acclimation function 200
Pressure axis 205
Time axis 210
Pre-ramp(Tpr)/snooze period(Tsnooze) 215
Ramp period (Tramp1) 220
Therapy period 225
Acclimation sleep period (Tsleep) 227
Therapeutic pressure (TP) 230
Acclimated daily therapeutic pressure for day 1 (TPdaily) 231
Acclimated daily therapeutic pressure for subsequent day (TPdaily) 232
Acclimated daily therapeutic pressure for last day (TPdaily) 233
Pressure/time curve for snooze function 235
Pre-snooze period (Tps) 240
Snooze sleep period (Tds) 245
First snooze trigger 250
Second snooze trigger 255
Pressure/time curve for snooze function with shorter ramp period 260
Third snooze trigger 265
Second ramp period (Tramp2) 270
Acclimation method implemented in PAP system 1000
Steps for acclimation method 1005-1080
Snooze method implemented in PAP system 2000
Steps for snooze method 2002-2082
Acclimation and snooze method implemented in PAP system 3000
Steps unique to acclimation and snooze method 3005-3040

FIG. 1A illustrates a PAP system, with a sleep apnea mask 10 worn by a patient 30, the mask 10 is connected to a blower 20 by a hose 15. The blower 20 may have several components, including a processor 31 connected to a display/touchscreen 35, an air pump 32 and a flow sensor 33 (see FIG. 1B). The processor 31 may also have a card reader 34A and antenna/transceiver 34B that may be used for programming, wireless programming, wireless control, wireless monitoring, and storage.

controls the operation of the blower 20. Many of the methods described herein would be implemented by the blower 20.

The patient mask designs previously disclosed in the related applications cited above, allows a patient to wear the mask and comfortably breathe when the blower is not delivering pressure. This mask design is a marked difference from the conventional PAP systems that require a continuously open flow path, from the blower to the patient so that the patient's expired breath, which could inadvertently travel into the hose to be re-breathed, is blown free of the hose and is vented into the room through the continuously-leaking vent in the mask or hose assembly. Should a PAP blower fail to provide a continuous flow of positive pressure air, there is a real risk that expired, CO2 laden breath will enter the hose and be re-breathed on the next user inhalation. This is a known condition called “CO2 rebreathing”, which can have harmful effects on the patient. When CO2 levels are elevated in the body it is known as hypercapnia. Rebreathing CO2 can lead to increased blood pressure, headaches, muscle twitches, rapid heart rate, chest pain, confusion, and fatigue. To mitigate this, users of conventional PAP masks are instructed to wear the mask with the blower set to at least its minimal setting, which is generally 4 [cm H2O] in air pressure. A pressure setting of 4 [cm H2O] corresponds to a typical continuous venting flow rate of 20 [lpm].

Engineering testing of the patient mask designs previously disclosed in the related applications cited above has shown particularly favorable results in reducing the accumulation of CO2 in the mask and airway. Testing has been conducted in accordance with a standard: ISO 17510_2015 Medical Devices-Sleep apnoea breathing therapy—Masks and application accessories Therapy Annex F “CO2 Rebreathing”. Testing revealed that in one of the worst case test conditions, with the blower off and the hose detached from the blower, the increase in intra-mask CO2 with the FRESCA embodiment was only 8-11%. This is considered a worst case condition for two reasons: 1) it is a configuration in which there is no airflow being delivered through the hose to flush out expired breath and 2) the hose is “open” at the farthest end from the patient which promotes migration of a patient's breath down the hose.

This testing confirms that the acclimation and snooze method described herein can be best utilized with the previously disclosed mask designs because the methods optimally have a period of zero pressure from the blower before any pressure is delivered. This is simply not possible with conventional PAP systems.

### Acclimation Method for Use in Pap System

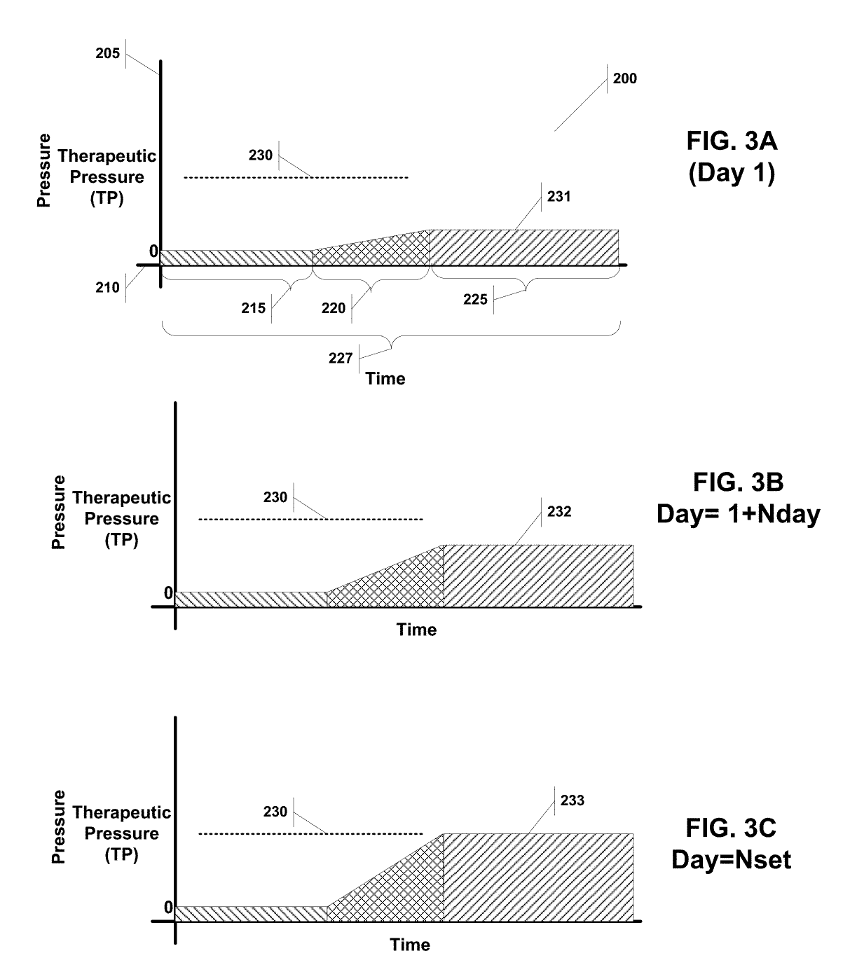

FIGS. 3A, 3B and 3C graphically illustrate pressure/time curves 200 for an acclimation method to assist a patient with PAP compliance. Each curve is plotted against a pressure axis 205 and a time axis 210. The pressure is maintained at zero for a pre-ramp period (Tpr), followed by a ramp period (Tramp1) characterized by gradually increasing the air pressure over Tramp1 until a plateau is reached at a desired pressure, which then marks the therapy period 225. The difference between the curves, is that the acclimated daily therapeutic pressure for day one 231 (FIG. 3A) is less than that of the subsequent day 232 (FIG. 3B), which is less than the last day 233 (FIG. 3C). In other words, the acclimation method increases the daily therapeutic pressure (TPdaily) gradually on a daily basis until the ultimate therapeutic pressure (TP) 230 is reached. The acclimation period (Nset) may be set for as little as three days, but is advantageously set to approximately ten to fourteen days.

The curves also show an acclimation sleep period (Tsleep) 227, which is used to confirm that the patient has experienced enough of the therapy period 235 for a particular day, such that the system can then increase the daily pressure for the next day. If, for example, the patient turns off the blower before Tsleep has elapsed on day 3, then the system will implement the same pressure for day 3 on day 4 until enough time has accrued to count as one complete Tsleep period. Alternatively, the system will only count days during which Tsleep has been achieved against the acclimation period.

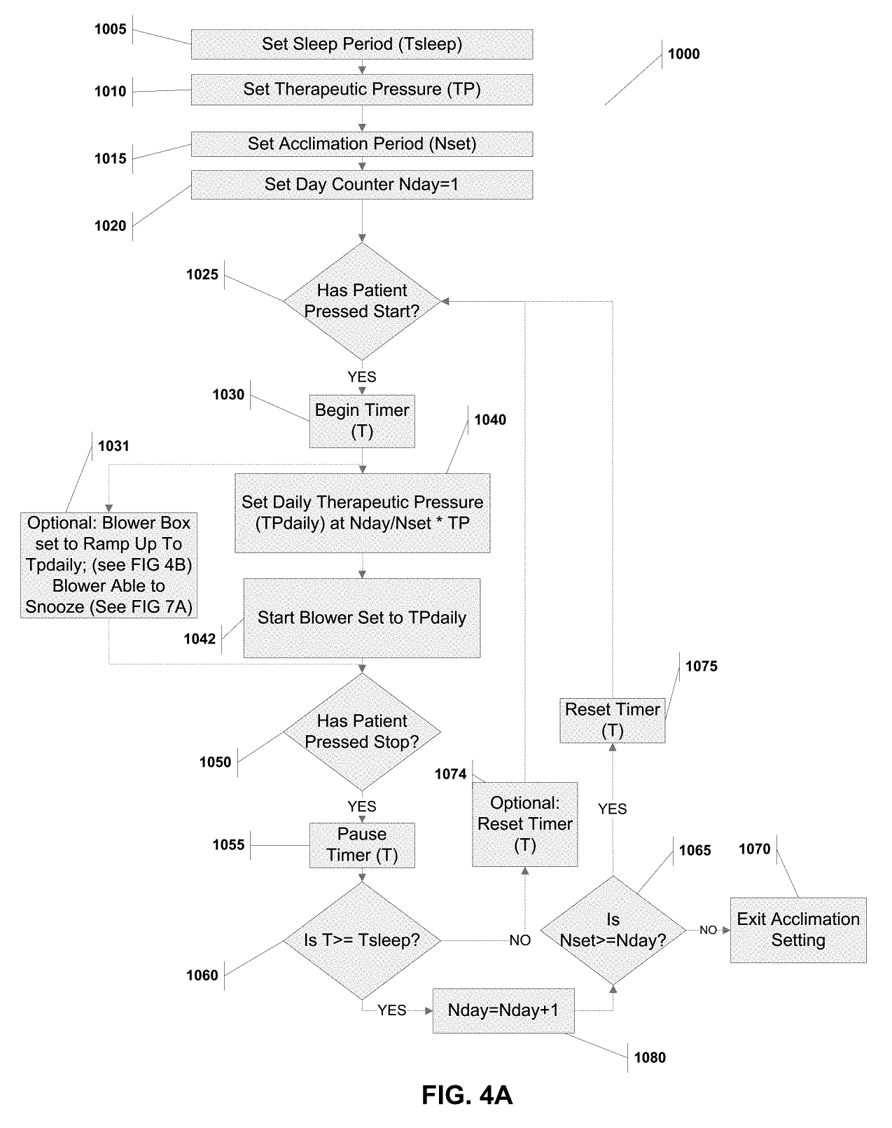

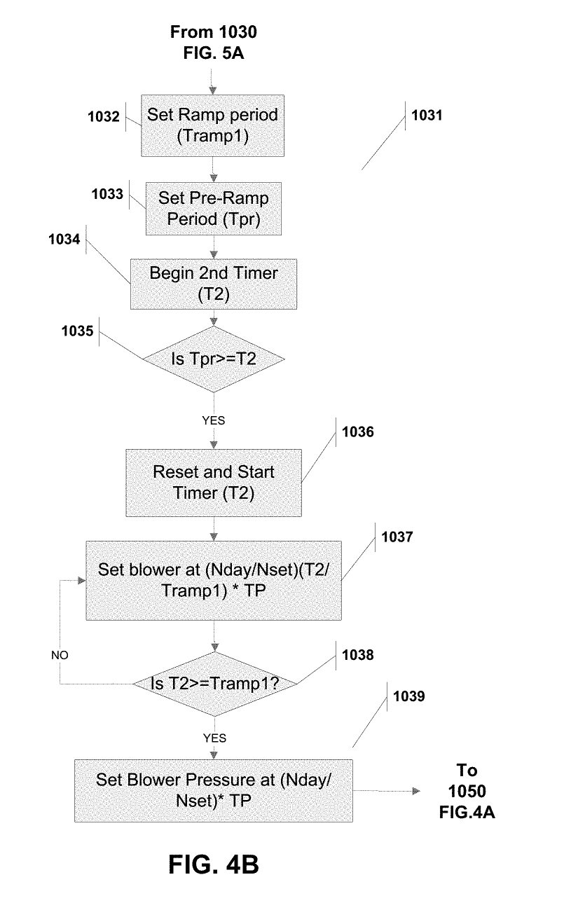

FIGS. 4A and 4B provide a flowchart with the steps for implementing the acclimation method 1000 on a blower processor. Steps 1005, 1010, and 1015 set the initial values of the acclimation sleep period (Tsleep), ultimate therapeutic pressure (TP) and acclimation period (Nset). A user such as a patient or physician would input these values based on the desired therapy.

The method 1000 then sets a day counter to 1 at step 1020 and waits for the patient to press start at step 1025. Once the patient presses start, the timer (T) is started and the air pump is set at the daily therapeutic pressure (TPdaily) defined as (Nday/Nset)×TP (steps 1030, 1040 and 1042. If the patient presses stop, the method pauses the timer T, and determines if the timer exceeds the acclimation sleep period (Tsleep) (steps 1050, 1055 and 1060). If it does exceed, the day counter (Nday) is incremented (step 1080) and the method determines if the entire acclimation period (Nset) has been reached (step 1065). If the acclimation period has been reached, then the acclimation method is exited at step 1070. If however, the acclimation period has not yet been reach, then the method resets the timer (T) (step 1075) and returns to waiting for the patient to press start (step 1025).

If back at step 1060, the timer does not exceed the acclimation sleep period (Tsleep), then the method may or may not reset the timer (step 1074) and does not increment the day counter (Nday), but does return to waiting for the patient to press start (step 1025). By not increasing the day counter (Nday), the patient does not get credit for the previous day's use of the PAP because the patient did not use it long enough. Also, if the method includes the optional timer (T) reset in step 1074, each time the patient shorts the acclimation sleep period (Tsleep), he will have to start over in order to have sufficient time to count the day against the acclimation period (Nset). This, however, may not be advantageous to progressing patients through the acclimation procedure; so instead the optional step 1074 may be omitted. This would then provide the patient credit for PAP time used in a previous session that did not achieve Tsleep. So for example, if in day 4 the patient uses the PAP system for 5 hours and Tsleep is set for 6 hours, then the method will not increment the day counter (Nday). However, the patient upon restarting the PAP system the next day will begin the timer at 5 hours and at the same daily therapeutic pressure (TPdaily) as the previous day, but will need only one additional hour to increment in to the next day in the acclimation period.

The acclimation method 1000 may optionally have a ramp up feature 1031 and pre-ramp period as shown graphically in FIGS. 3A-3C. The subroutine to implement this is shown in FIG. 4B. The patient or physician would set a ramp period (Tramp1) and a pre-ramp period (Tpr) at steps 1032 and 1033. The method allows the pre-ramp period (Tpr) to elapse (steps 1034 and 1035) and then ramps up the pressure over the ramp period (Tramp1) until it plateaus at TPdaily defined as ((Nday/Nset)×TP) (steps 1036, 1037, 1038 and 1039). The system then returns to step 1050 and continues as described before.

A processor implementing the acclimation method 1000 may set Tsleep, TP, Nset, Tramp1, and Tpr based on the user's (e.g. patient or physician) input. Optimal values for Tsleep is between 4 and 8 hours, for TP is between 4 and 20 CM H20, for Nset is between 3 and 14 days, and for either or both of Tramp1 and Tpr is between 0.25 and 1.5 hours. And as discussed below, the processor may adjust TP based on predicted patient breathing patterns.

It should be noted that there are a multitude of acclimation scenarios that can be contemplated, including a series of nights where the pressure does not increase.

It is possible to apply the system as described above at non-therapeutic pressure settings due to the nature of sleep apnea. It is a chronic condition that is often untreated for months or years. Using a period of days or weeks to allow an acclimation period is an acceptable trade off if it allows the user to be more compliant with or tolerant of the needed chronic therapy. The risks associated with PAP treatment are generally considered long-term, accumulated risks, so it has been found generally acceptable to sacrifice some minor part of efficacy in return for better, sustained long-term compliance.

### Snooze Method for Use in Pap System

The patient mask designs previously disclosed in the related applications cited above system can be worn with the blower in an off condition without the risk of CO2 rebreathing. Additionally, it is disclosed that the user can wear the system while awake and going to sleep with the blower in an off condition, set to activate spontaneously after a set period of time or after detecting the user has fallen asleep based on monitoring the breath rate, tidal volume, or both. The concept of having the blower in the off condition can be expanded to allow the user to “snooze” the system by activating a snooze feature. The intention of this feature is to allow the user to re-enter the mode of the blower in an off condition if he or she wakes sometime during the night and wishes to return to initiate sleeping in the more comfortable mode of operation. This could also be useful for patients who find that they have to utilize the restroom one or more times during the night, and would like to return to sleep comfortably using a “blower off” condition for a pre-specified period, such as 5 to 10 minutes. This would be different from the ramp, as it would allow the user to return to therapy more quickly than the ramp, yet still provide the comfort benefit of the blower off condition while returning to sleep. The snooze feature would have user-specified or pre-set time periods for return to full therapeutic pressure.

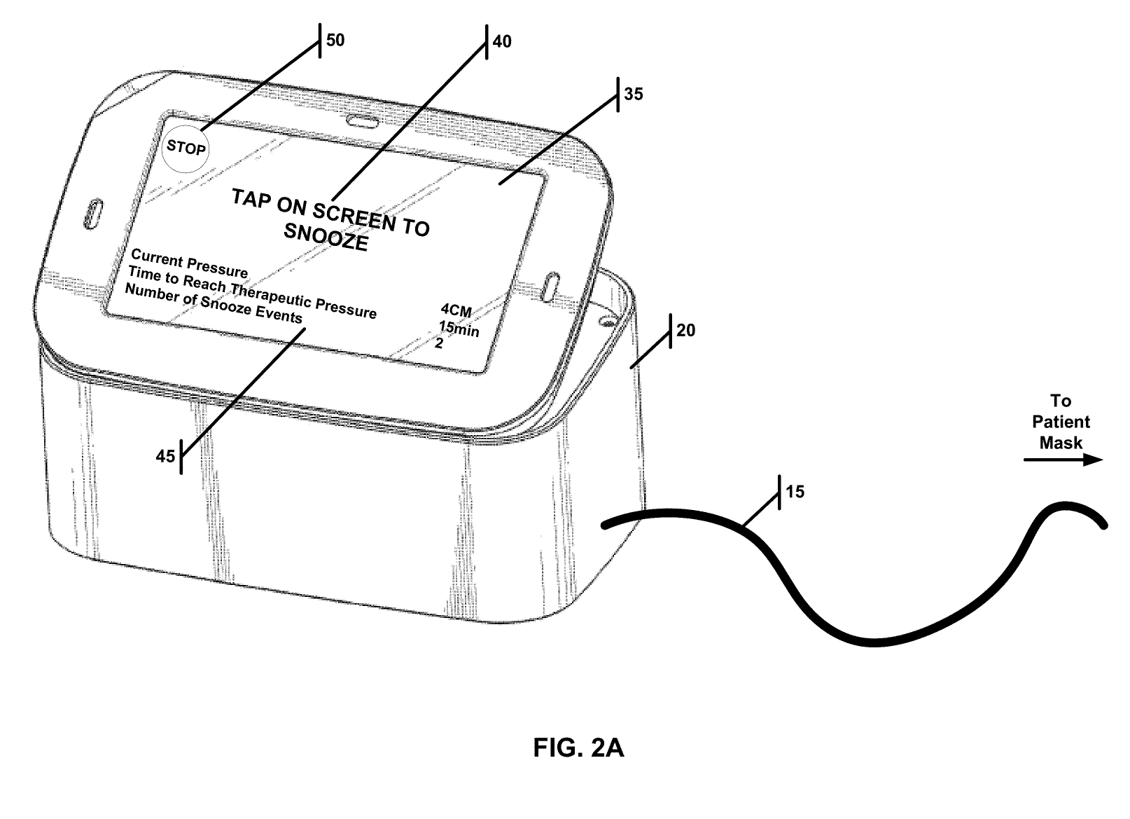

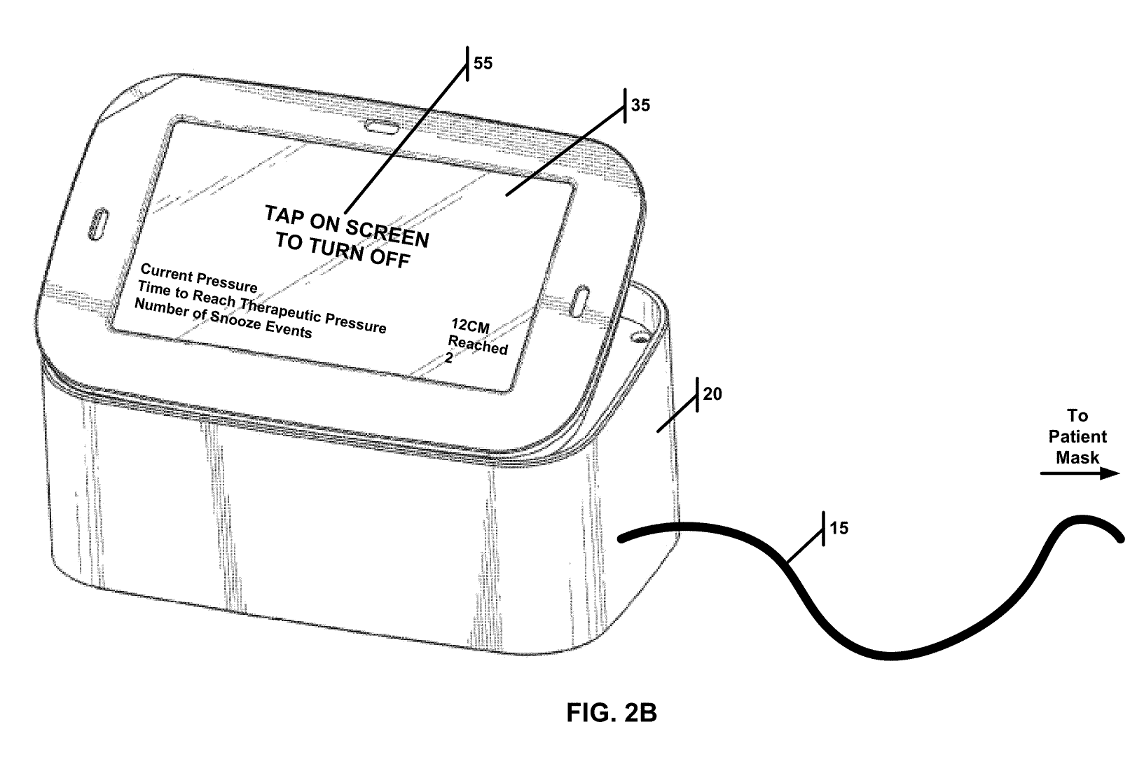

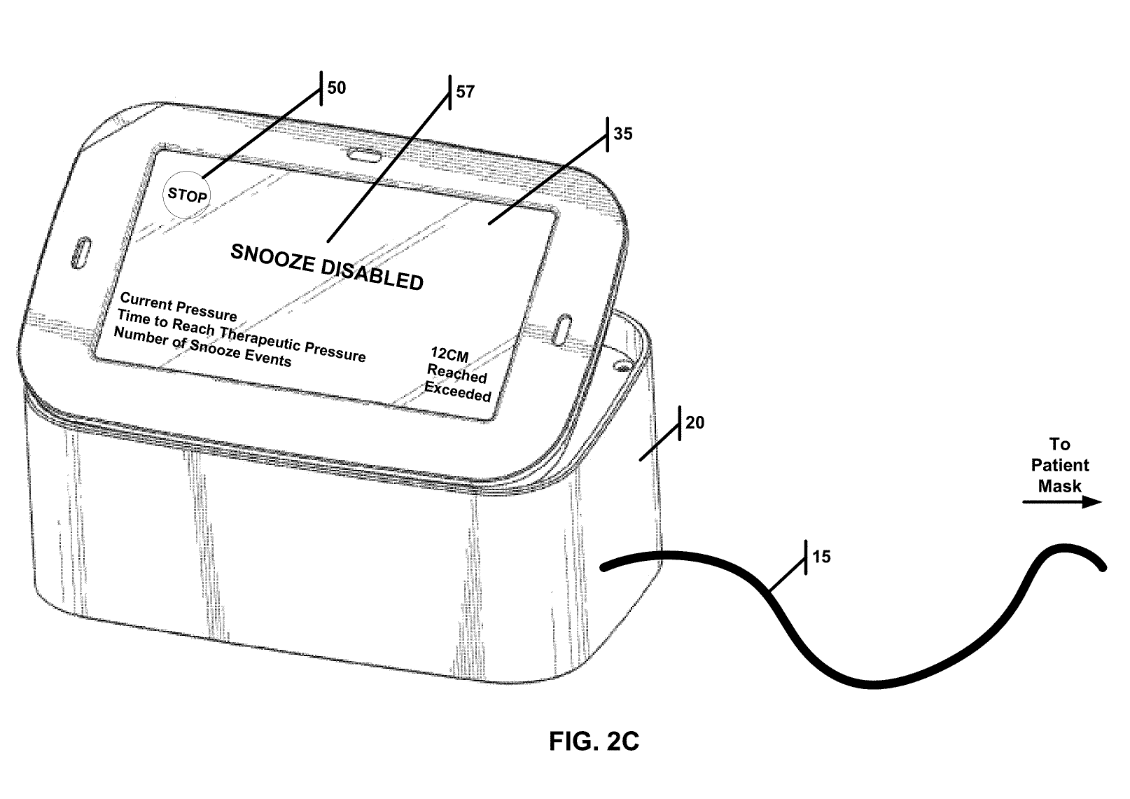

FIGS. 2A, 2B and 2C illustrate a blower 20 with a display/touchscreen 35. The display/touchscreen can display the current blower setting 45. FIG. 2A illustrates the blower box 20 in a snooze configuration. Specifically, virtually the entire display/touchscreen 35 may be used as the snooze start/stop button 40. The exception is a small stop button 50 in the corner of the touchscreen 35. When in the snooze configuration, the patient may trigger a snooze by pressing the touchscreen 35 almost indiscriminately. This is helpful given that the patient would likely be groggy, and in the dark when triggering a snooze. If, however, the patient would like to turn off the blower box, he would need to act more deliberate and locate the smaller stop button 50. In essence, during the snooze configuration the snooze button is larger and easy to trigger.

When the patient has either slept long enough, the blower 20 may disable snooze transition out of the snooze configuration as shown in FIG. 2B. Here, the touchscreen 35 has a large stop button comprising the whole screen. A patient may press anywhere on the touchscreen 35 to turn off the blower 20.

FIG. 2C illustrates a third configuration, where the patient has not slept long enough and has triggered too many snooze events. It is not advantageous to allow a patient to trigger too many snooze events because this would be counterproductive. The patient could be allowed a maximum number of snooze events, after which a snooze trigger is ignored and the touchscreen 35 displays a snooze disabled notification 57.

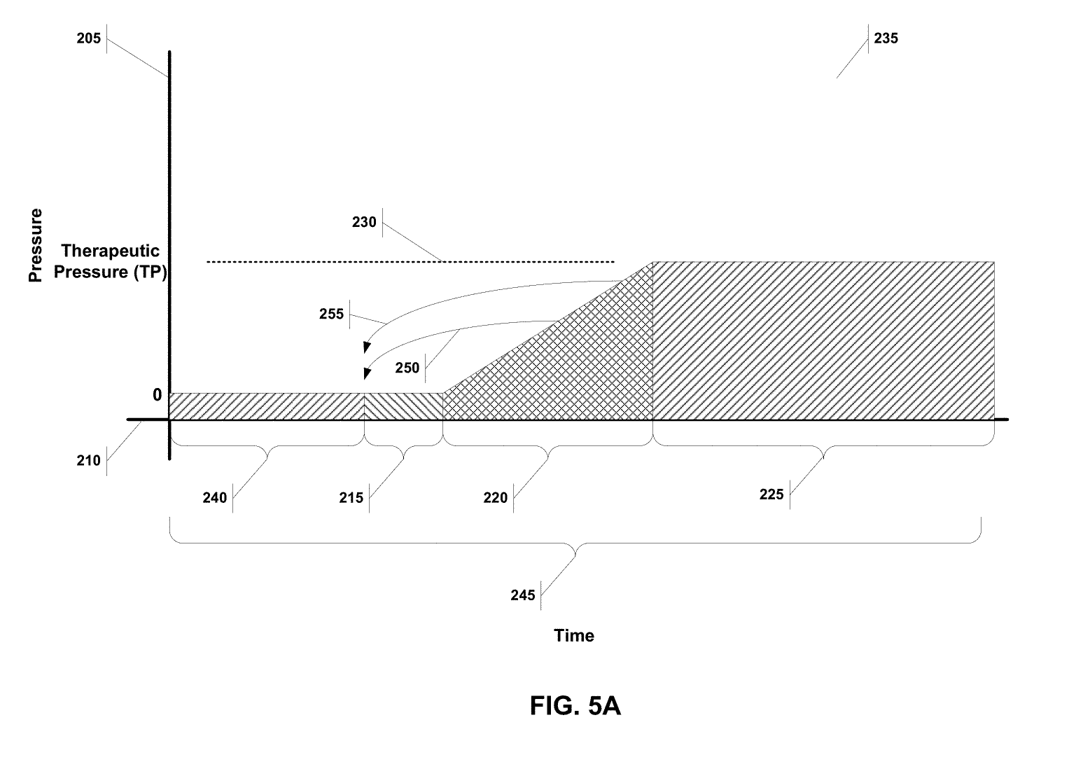

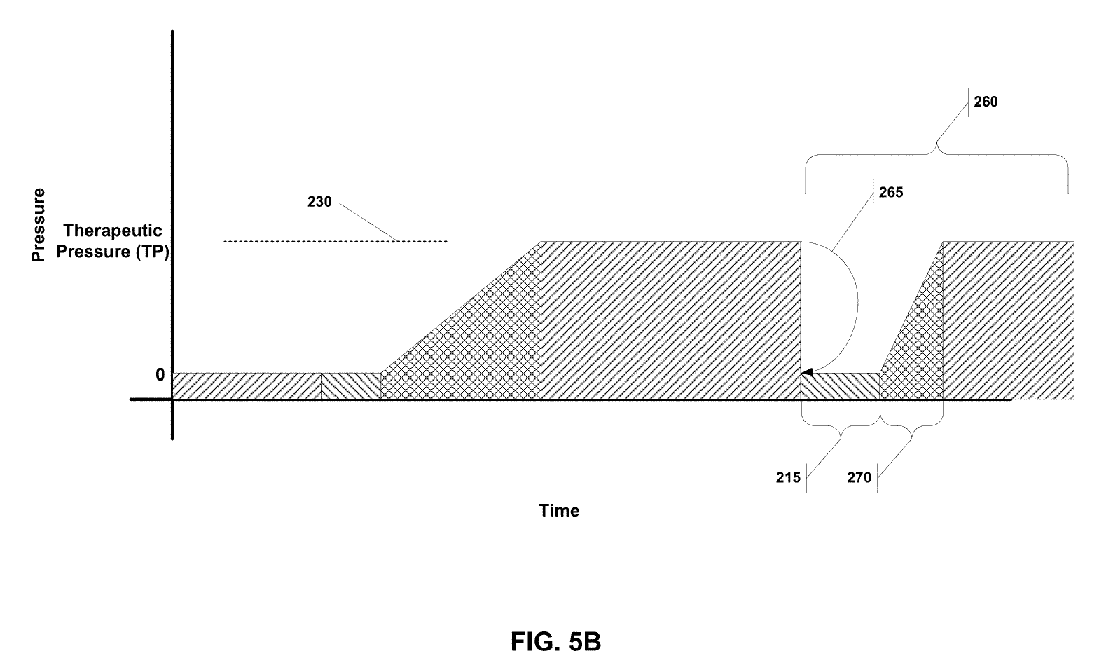

FIG. 5A illustrates Pressure/time curve for snooze function 235. This curve has some of the same features shown in FIGS. 3A-3C, but also includes a pre-snooze period (Tps) 240 and snooze sleep period (Tds). Arrows 250 and 255 are a first and second snooze triggers that return the blower to beginning of the snooze period (Tsnooze). If however, the patient waits until the therapy period 225 to press snooze, as shown in FIG. 5B as arrow 265, the method may implement a shorter ramp period 270 (Tramp2), as shown in pressure/time curve for snooze function with shorter ramp period 260. The rationale for having a shorter ramp period 270 (Tramp2) is that the patient has already achieve therapy pressure, and therefore is likely comfortable enough to reach that pressure again quickly, thereby giving the patient longer periods in the therapy period 225.

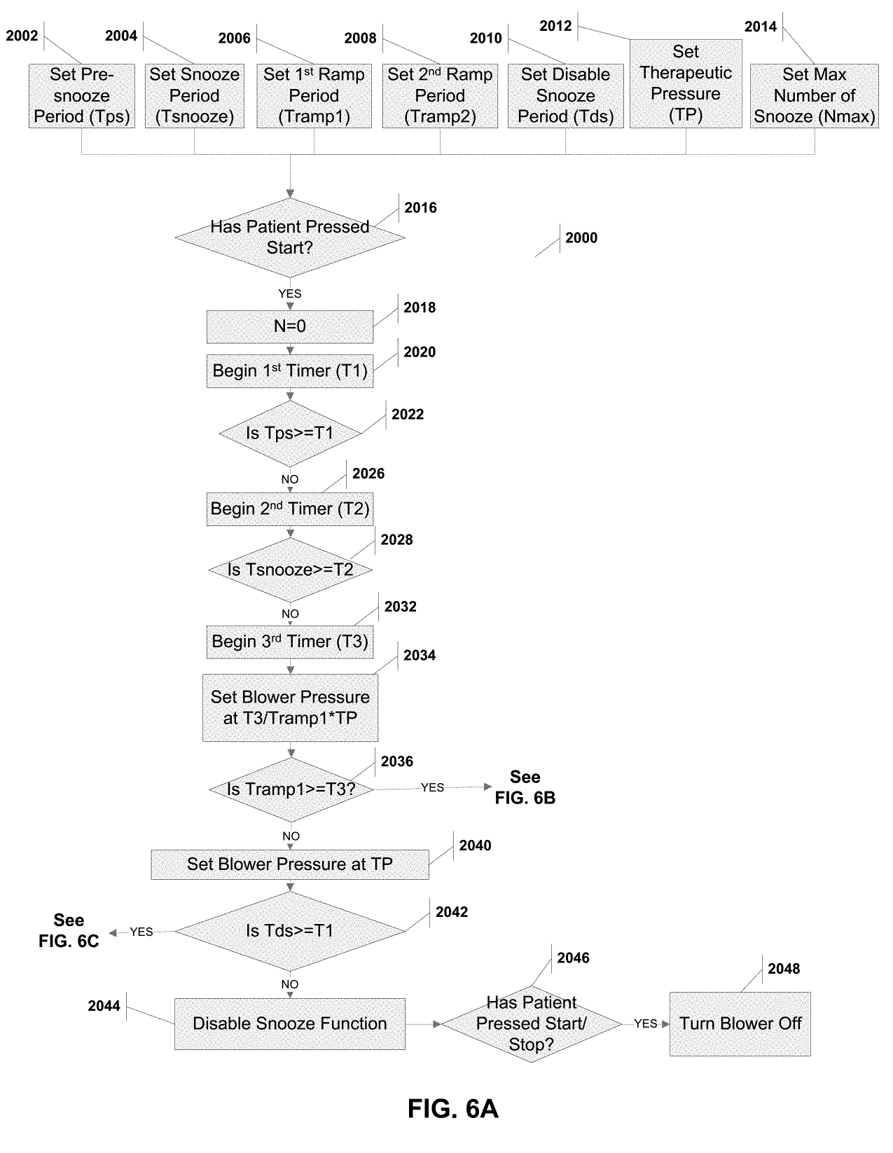

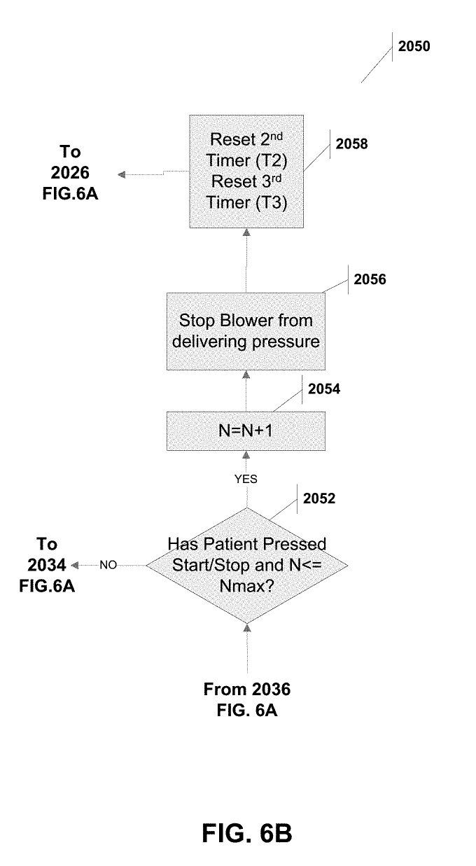

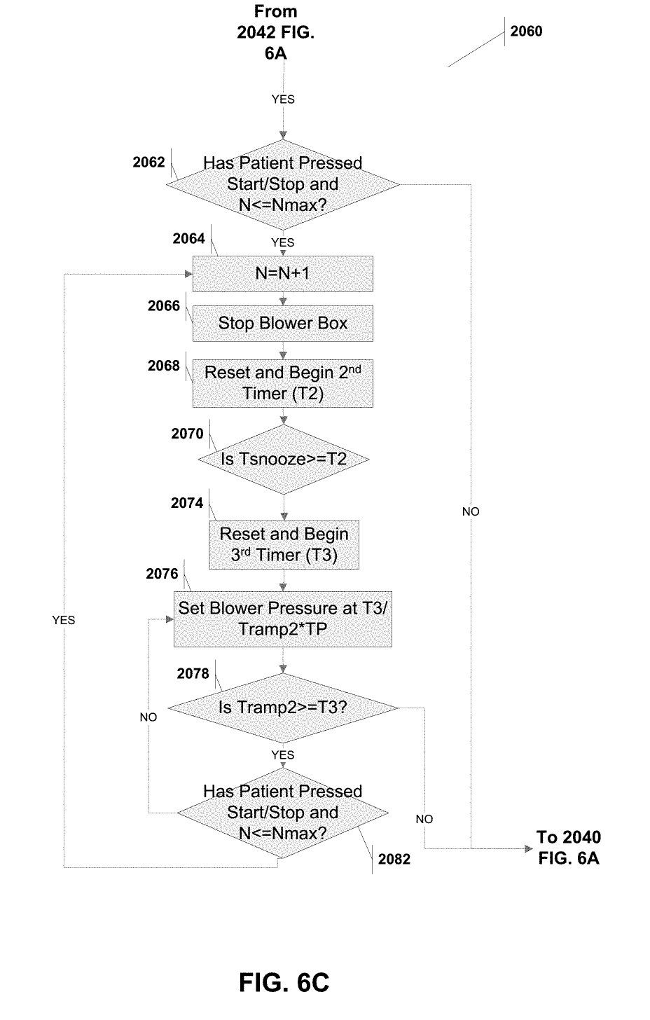

FIGS. 6A, 6B and 6C provide a flowchart with the steps for implementing the snooze method 2000 on a blower processor. Steps 2002 through 2014 sets the initial values of the pre-snooze period (Tps), the snooze period (Tsnooze), the first ramp period (Tramp 1), the second ramp period (Tramp2), the disable snooze period (Tds), the therapeutic pressure (TP), and the maximum number of snooze triggers (Nmax). A user such as a patient or a physician would input these values based on the desired therapy.

The method 2000 waits for the patient to press the start/stop button at step 2016. After such a press, the method 2000 set a counter N to 0 (step 2018) to monitor that the patient has not exceed the maximum number of snooze triggers (Nmax). This counter N may be set at the initiation stage of the method (i.e., prior to step 2016). The pre-snooze period (Tps) is allowed to elapse (steps 2020, 2022) as is the snooze period (Tsnooze) (steps 2026, 2028). Steps 2032 through 2040 gradually ramp up the blower pressure over the ramp period (Tramp1) until it plateaus at the therapeutic pressure TP.

If the patient has used the blower a sufficient amount of time (i.e., Tds), then the method disables the snooze function (steps 2042 and 2044). Any press of the blower buttons subsequent to this is considered an intention to turn off the blower (steps 2046, 2048). These two states (i.e., prior to Tds and after) are shown in FIGS. 2A and 2B, respectively.

FIG. 6B illustrates a subroutine 2050 that is implemented when the method 2000 is still in the initial ramp period (Tramp1). During this period, the method 200 checks if the patient has pressed the start/stop button (step 2052). If the patient has, and the patient has not exceeded the maximum number of snooze triggers (Nmax), then the counter N is incremented, the blower stops delivering pressure and timers are reset (steps 2054, 2056 and 2058), and the method 2000 returns to step 2026. This is shown by snooze triggers 250 and 255 in FIG. 5A.

FIG. 6C illustrates a subroutine 2060 that is implemented when the method 200 has past the initial ramp period (Tramp1). Any press of the start/stop button at this time would implement a shorter ramp period (Tramp2), as shown by the snooze trigger 265 in FIG. 5B. The method 2000 checks if the patient has pressed the start/stop button (step 2062). If the patient has, and the patient has not exceeded the maximum number of snooze triggers (Nmax), then the counter N is incremented, the blower stops delivering pressure and the second timer (T2) is reset and started (steps 2064, 2066 and 2068). The snooze period is allowed to elapse (step 2070) and in steps 2074 and 2078 the blower pressure is gradually ramp up over the ramp period (Tramp2) until it plateaus at the therapeutic pressure TP (step 2040). If during this period, the patient has pressed the start/stop button and has not exceeded the maximum number of snooze triggers (step 2082), then the method 2000 returns to step 2064 (i.e., the pre-snooze time position followed by a shorter ramp period).

It should be noted that in the even the patient has exceeded the maximum number of triggers Nmax, then the method effectively ignores the patients snooze trigger (see 2052, 2062, 2082). This state is illustrated in FIG. 2C.

A processor implementing the snooze method 2000 may set Tsnooze, Tramp1, Tramp2, Tds, Tps, TP and Nmax based on the user's (e.g. patient or physician) input. Optimal values for Tsnooze, Tramp 1, Tramp 2 and Tps is between 0.1 and 1.5 hours, for Tds is between 4 and 8 hours, TP is between 4 and 20 CM H20, for Nmax is between 3 and 6. And as discussed below, the processor may adjust TP based on predicted patient breathing patterns.

### Acclimation and Snooze Method for Use in Pap System

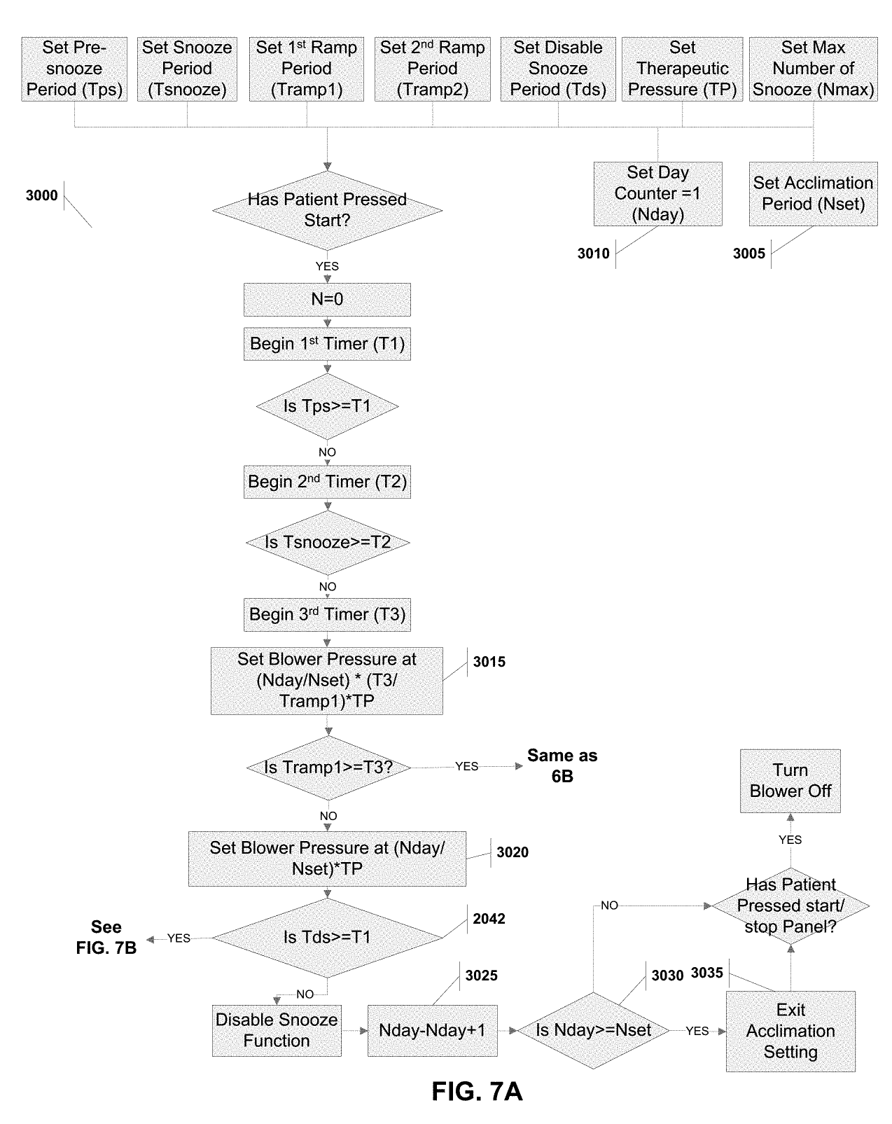

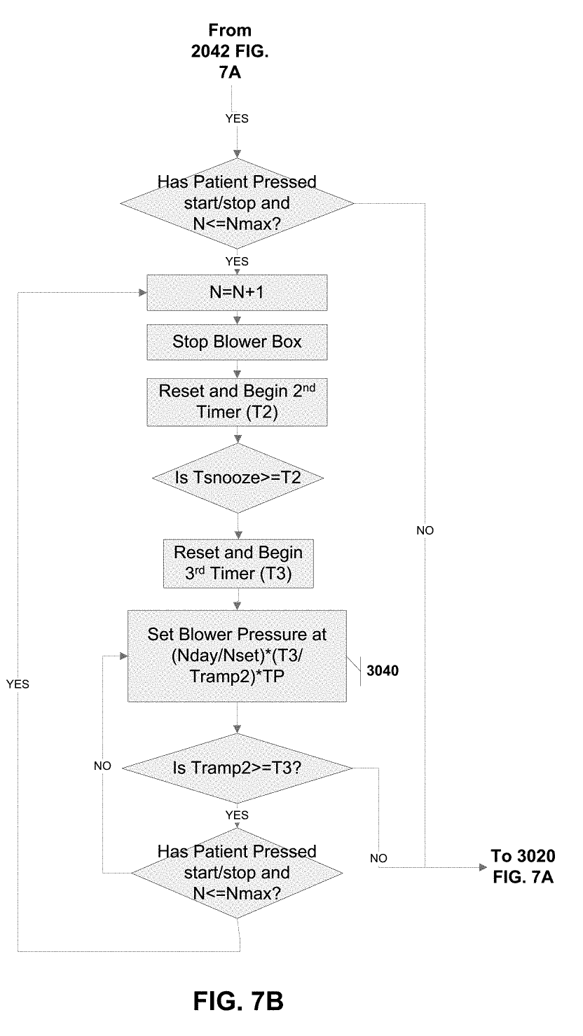

FIGS. 7A and 7B illustrate a method for combining both the acclimation and snooze methods just described. The method is substantially similar to that of the snooze method 2000 described with reference to FIGS. 6A-6C. There are, however, a few steps/functions that need to be added to include the acclimation features. For simplicity, the new steps/functions have been labeled with new part numbers, while the steps/functions that remain the same have not been labeled.

Steps 3005 and 3010 set the day counter (Nday) and an acclimation period (Nset). In steps 3015, 3020 and 3040, the blower is set to a pressure that is reduced by the fraction of (Nday/Nset) from the previous snooze only method. If the snooze period is exceeded (step 2042) then the method will also increment the day counter Nday (step 3025) and check if the acclimation setting should be exited in steps 3030 and 3035.

Finally, the therapeutic pressure (TP) described above need not be static, but may change with treatment efficacy. If, for example, the patient is not experiencing an apnea or hypopnea (shallow breathing) event while on the current TP, then the system may reduce the TP, thus providing more comfort to the patient. Conversely, if under the current TP the patient still experiences apnea or hypopnea, then the system could increase the TP. The blower box 20 may have a flow sensor 33 connected to the processor 31 (See FIG. 1B). Because of the design of the patient masks disclosed in the related applications cited above, the blower box 20 can detect the regular breathing cycle of the patient. Specifically, the air flow through the hose 15 during a patient exhalation will be near zero, and during inhalation will be measurably and reliably higher. The processor 31, by use of the air flow sensor, may therefore measure these cyclical airflows (over a first period) and measure or predict the regular breathing cycle of the patient. A deviation from the measurement or prediction (over a second period) may be an apnea or hypopnea event. For example, an apnea event may be defined as less than 10% of the expected airflow over 10 seconds, and a hypopnea may be between 10% and 70% of expected airflow over 10 seconds. Any such event may require an adjustment to the TP.

To illustrate, the TP may be set for the snooze method at 12 CM H20. If the processor 31 detects that over the course of several hours that no event has occurred, the processor 31 may lower the TP to 10 CM H20 and again monitor the patient breathing patterns. If, however the processor 31 detects an apnea event, then it may increase the TP to 15 CM H20 and continuing monitoring. Likewise, a hypopnea event may cause the processor 31 to increase the TP, but perhaps not as severe as an apnea event. The processor 31 may periodically adjust the TP to arrive at the minimum necessary TP to prevent detected events. U.S. application Ser. No. 15/334,243 filed Oct. 15, 2016 titled “Apparatus, Systems, and Methods For Treating Obstructive Sleep Apnea”, incorporated herein by reference, includes an additional description of techniques and structures that may be used to detect apnea of hypopnea, and this can be used by the processor to adjust TP.

Various settings have been described as selectable by the user (patient or physician). These may be set at the time of prescription of the device. They may be set periodically such as nightly. Some settings, such as TP may be configured to be only selectable and settable by the physician. Settings may be adjusted through the touchscreen of the blower, reprogramming by inserting of a memory card in the card reader with software updates, and even through remote control such as “Bluetooth” interaction with a portable phone and dedicated application. These functions may be implemented using the card reader 34A and the antenna/transceiver 34B shown in FIG. 1B.

Although exemplary embodiments and applications of the invention have been described herein including as described above and shown in the included example Figures, there is no intention that the invention be limited to these exemplary embodiments and applications or to the manner in which the exemplary embodiments and applications operate or are described herein. Indeed, many variations and modifications to the exemplary embodiments are possible as would be apparent to a person of ordinary skill in the art. The invention may include any device, structure, method, or functionality, as long as the resulting device, system or method falls within the scope of one of the claims that are allowed by the patent office based on this or any related patent application.

1. A blower for the treatment of a patient's sleep apnea, the blower comprising:
a start/stop button;
an air pump;
a processor connected to the start/stop button and air pump, the processor configured to perform an snooze procedure with the following steps:
a. set a snooze period (Tsnooze), set ramp period (Tramp1), and set an ultimate therapeutic pressure (TP),
b. When the patient presses the start/stop button:
i. allow Tsnooze to elapse, start a timer (T3), and set the air pump to deliver air pressure at a pressure based on ((T3/Tramp1)×TP);
ii. If during step (b)(i) patient presses the start/stop button, then reset T3, stop delivering air pressure, and return to step (b)(i).

2. The blower of claim 1, wherein the processor is further configured to perform the following step: if T3>=Tramp 1 then set the air pump to deliver air pressure at a pressure based on TP;

3. The blower of claim 2, wherein the processor is further configured to perform the following step: if patient presses the start/stop button when T3>=Tramp1 then reset T3, stop delivering air pressure and return to step (b)(i).

4. The blower of claim 2, wherein the processor is further configured to perform the following steps:
set a snooze disable period (Tds),
after step (b) but before (b)(i), start a timer (T1);
if patient presses the start/stop button and Tds>=T1, then stop blower and exit snooze procedure.

5. The blower of claim 2, further comprising a touchscreen connected to the processor, wherein the processor is further configured to perform the following steps:
set a snooze disable period (Tds),
after step (b) but before (b)(i), start a timer (T1);
while Tds>=T1 the start/stop button comprises a snooze button displayed on the touch screen;
while Tds<T1 the start/stop button comprises a stop button displayed on the touch screen.

6. The blower of claim 1, wherein the processor is further configured to perform the following steps:
set a pre-snooze period (Tps);
after step (b) but before (b)(i), allow Tps to elapse.

7. The blower of claim 1, wherein the processor is further configured to perform the following steps:
set a max number of snooze triggers (Nmax);
set counter N=0;
each time patient presses the start/stop button, increase N;
when N>=Nmax, then ignore step (b)(ii).

8. The blower of claim 2, wherein the processor is further configured to perform the following steps:
set a second ramp period (Tramp2);
if patient presses the start/stop button when T3>=Tramp1 then:
i. allow Tsnooze to elapse, and reset and start T3
ii. set the air pump to deliver air pressure at a pressure based on ((T3/Tramp2)×TP);
iii. if T3>=Tramp2 then set the air pump to deliver air pressure at a pressure based on TP.

9. The blower of claim 1, further comprising a flow detector connected to the processor and adapted to measure the airflow delivered to the patient, wherein the processor is further configured to perform the following steps:
measure the air flow over a first period;
measure the air flow over a second period;
detect an apnea or hypopnea event based on a deviation between the measured air flows;
adjust the TP based on the detected apnea or hypopnea event.

10. The blower of claim 1, wherein the processor sets Tsnooze, Tramp1 and TP based on a user's input.

11. The blower of claim 4, wherein the processor sets Tds based on a user's input.

12. The blower of claim 6, wherein the processor sets Tps based on a user's input.

13. The blower of claim 7, wherein the processor sets Nmax based on a user's input.

14. The blower of claim 8, wherein the processor sets Tramp2 based on a user's input.

15. The blower of claim 14, wherein either or both of Tramp 1 and Tramp 2 are set between 0.25 and 1.5 hours.

16. The blower of claim 1, wherein either or both of Tsnooze and Tramp 1 is set between 0.1 and 1.5 hours.

17. The blower of claim 1, wherein TP is set between 4 and 20 CM H20.

18. The blower of claim 4, wherein Tds is set between 4 and 8 hours.

19. The blower of claim 5, wherein Tps is set between 0.1 and 1.5 hours.

20. The blower of claim 7, wherein Nmax is set between 3 and 6.

21. A blower for the treatment of a patient's sleep apnea, the blower comprising:
a start/stop button;
an air pump;
a processor connected to the start/stop button and air pump, the processor configured to perform an snooze procedure with the following steps:
a. receive a snooze period (Tsnooze), set ramp period (Tramp1), and an ultimate therapeutic pressure (TP),
b. When the patient presses the start/stop button:
i. allow Tsnooze to elapse;
ii. then deliver air pressure ramped up to and plateaued at the value of TP over period Tramp 1;
c. If during steps (b)(i) or (b)(ii) the patient presses the start/stop button, then:
i. stop delivering air pressure and return to step (b)(i).

22. The blower of claim 20, wherein step (a) further comprises receiving a max number of snooze triggers (Nmax), and step (c) further comprises before performing step (c)(i), determining the number of times the patient presses the start/stop button exceeds Nmax, if so then return to step (b)(ii).

23. The blower of claim 20, wherein step (a) further comprises receiving a pre-snooze period (Tps); and step (b) further comprises before performing step (b)(i), allowing Tps to elapse.

24. The blower of claim 20, wherein step (a) further comprises receiving a snooze disable period (Tds); and step (c) further comprises before performing step (c)(i), determining if Tds has elapse since the patient presses the start/stop button in step (b), and if so stop delivering air pressure and exit snooze procedure.

25. The blower of claim 21, further comprising a flow detector connected to the processor and adapted to measure the airflow delivered to the patient, wherein the processor is further configured to perform the following steps:
measure the air flow over a first period;
measure the air flow over a second period;
detect an apnea or hypopnea event based on a deviation between the measured air flows;
adjust the TP based on the detected apnea or hypopnea event.
